(19) United States
(12) Patent Application Publication
(10) Pub. No.: US 2014/0012300 A1
(43) Pub. Date: Jan. 9, 2014

(54) Ultrasonic Wave Generator and Method of Lipolysis

(71) Applicant: Switech Medical AG, Kreuzlingen (CH)

(72) Inventors: Grob Nino, Kreuzlingen (CH); Axel Voss, Kreuzlingen (CH)

(73) Assignee: Switech Medical AG, Kreuzlingen (CH)

(21) Appl. No.: 14/021,979

(22) Filed: Sep. 9, 2013

Related U.S. Application Data
(63) Continuation of application No. 12/971,280, filed on Dec. 17, 2010, now abandoned.

(51) Int. Cl. A61B 18/04

(52) U.S. Cl. CPC A61B 18/04
USPC 606/169

(57) ABSTRACT

The invention relates to a device for generating an ultrasonic field having frequencies between 0.9 MHz and 4 MHz in an object, preferably in water or in human tissue, the device comprising a piezo element (1) affixed to a coupling plate (5), the coupling plate emitting the ultrasonic field (6) into the object, wherein the dimensions of the coupling plate are chosen such that the intensity of the ultrasonic field (6) emitted into the object is minimized in a depth deeper than 70 mm.

### TECHNICAL FIELD

The invention relates to a device for generating an ultrasonic field, in particular for the treatment of human tissue for lipolysis. Further the invention relates to a method of lipolysis.

### TECHNICAL BACKGROUND

The use of ultrasonic fields in human tissue is a well known technology which finds applications in medical imaging. Diagnostic ultrasonography, for example, is an ultrasound-based diagnostic imaging technique used for visualizing subcutaneous body structures including tendons, muscles, joints, vessels and internal organs for possible pathology or lesions. Obstetric sonography is commonly used during pregnancy and is widely recognized by the public.

Ultrasound is also known and publically perceived to be used in the fields of dental hygiene, for the treatment of cysts and tumors and for breaking up kidney stones by lithotripsy.

Ultrasonic lipolysis is also a known application of ultrasonic fields. However, in order to be in a position to apply a high intensity of ultrasonic energy into adipose tissue in order to provide an efficient lipolysis, but at the same time reducing the energy exposition of the remaining tissue and body of the patient, it is desirable to apply a strong ultrasonic field of a reduced depth to the adipose tissue.

In other words, for the application of lipolysis it is important that the ultrasonic field does not penetrate deeply into the tissue, but is accumulated in a near-zone, close to the skin. It is known to create an ultrasonic field in the near-zone and to avoid a deep penetration into the tissue by using focused wave generators. However such devices have the disadvantage of strongly limiting the area of the field in x- and y-direction, when the z-direction is directed towards the depth into the tissue. For applications where larger areas (approx. 50 sqmm to 10⁶ sqmm) need treatment, the known devices are not suitable.

It is desirable to apply the ultrasonic field over a relatively large area (approx. 50 sqmm to 10̂6 sqmm) to the patient in order to keep the times for treatment for the individual patient low. Furthermore, it is desirable to maintain a relatively homogeneous intensity distribution of the ultrasonic field over this area.

### DESCRIPTION OF THE DISCLOSURE

On the basis of the above, the problem to be solved is to provide a device that generates an ultrasonic field with a reduced penetration depth into the tissue and at the same maximizes and homogenizes the intensity of the field in a zone near to the skin.

This object is solved by a device for generating an ultrasonic field having frequencies between 0.9 MHz and 4 MHz in an object, preferably in water or in human adipose tissue, the device comprising a piezo element affixed to a coupling plate, the coupling plate emitting the ultrasonic field into the object. According to the invention, the dimensions and/or material of the coupling plate are chosen such that the intensity of the ultrasonic field emitted into the object is minimized in a depth deeper than 70 mm.

It has been found that by correctly choosing the dimensions of the coupling plate in relation to the piezo element, the intensity of the ultrasonic field emitted into the object can be minimized in a depth deeper than 70 mm. Accordingly, the intensity in the object in an area deeper than 70 mm below the object's surface, in particular below the skin, is minimized such that the energy of the ultrasonic field is only effective in the first 70 mm from the skin.

The effective penetration depth of the ultrasonic field is reduced and the far field of the ultrasonic field is substantially non-existent. These fields are effectively minimized below the limit of 3W/qcm (see DIN EN 61689:2007).

Furthermore, it has been found that the near field is substantially homogenized by means of a suitable choice of material and dimensions of the coupling plate. Accordingly, with the device provided, a large-sized area can be treated in a surface-near section of the skin, as it is advantageous in various medical applications such as lipolysis.

The term “ultrasonic” in principle refers to an acoustical wave having a frequency greater than the limit of human hearing, i.e. above 20 kHz.

Preferably, the dimensions and/or material of the coupling plate are chosen such that the intensity of the ultrasonic field emitted into the object is minimized in a depth deeper than 40 mm, preferably in a depth deeper than 30 mm. By the correct choice of the coupling plate in relation to the piezo element even more shallow penetration profiles can be achieved.

Preferably, the diameter of the coupling plate, the thickness of the coupling plate and/or the material of the coupling plate are dimensioned such that the intensity of the ultrasonic field emitted into the object is minimized in a depth deeper than 70 mm, preferably in a depth deeper than 40 mm, more preferred in a depth deeper than 30 mm. By simple choice of these characteristics of the coupling plate the desired profile of the intensity of the ultrasonic field can be achieved.

Preferably, the relation of the radial mode frequency to the serial mode frequency of the piezo element is set to 1.13-1.16, and/or the piezo element has a resonance frequency of between 0.9 and 4 MHz, and/or the piezo element has a diameter of between 20 and 70 mm, preferably between 25 and 50 mm, more preferably between 28 and 40 mm, most preferred of 30 mm, and/or the piezo element has a capacity of between 3,000 and 6,500 pF, preferably between 3,500 and 5,000 pF, most preferred between 3,600 and 4350 pF and/or the piezo element is of the lead-zirconate-titanate-type (PZT). By the choice of these dimensions and characteristics of the piezo element, it becomes possible to achieve the desired intensity distribution of the device. The lead-zirconate-titanate-type (PZT) piezo element may be used because it has been found that the piezo effect is rather strong for this type.

Preferably, the coupling plate is dimensioned such that at least two different frequencies are emitted from the coupling plate into the object when the piezo element is driven at a single frequency, the at least two frequencies emitted by the coupling plate preferably having a frequency difference of 5 to 100 kHz, more preferably a frequency difference of 10 to 50 kHz. Due to the at least two frequencies which are emitted by the coupling plate, destructive interferences of the ultrasonic waves of the two different frequencies can be observed in an area deeper than 70 mm. The emission of the two frequencies despite the piezo element being driven at a single frequency is attributed to the provision of the specific coupling plate. The superposition of the two frequencies leads to an interference of the two different waves resulting in beats (perceived as periodic variations in volume whose rate is the difference between the two frequencies) having a specific beat frequency. The beat frequency depends on the difference in frequencies which in itself depends on the specific choice of material and dimensions of the coupling plate. The denser the material of the coupling plate is the higher is the beat frequency.

Preferably, the coupling plate comprises a synthetic material, a plastics material, a composite material and/or a metal, and is preferably made from aluminum, more preferably from aluminum 7075, the material of the coupling plate preferably having a sound velocity of between 3,000 and 6,000 m/s. By the proper choice of the material, the effect of the significant reduction of the intensity of the ultrasonic field in a penetration depth deeper than 70 mm can be achieved.

In order to achieve the effects described above, the coupling plate preferably has a thickness of between 3 and 15 mm, preferably 5 to 12 mm, more preferably between 6 and 9 mm, even more preferred between 7 and 8 mm, most preferred between 7.0 and 7.3 mm, and/or the coupling plate preferably has a circular shape and has a diameter of between 35 and 95 mm, preferably 45 and 85 mm, more preferably between 55 and 80 mm, most preferred between 60 and 75 mm, and/or the coupling plate preferably has a circular shape and has an active diameter of between 25 and 60 mm, preferably between 35 and 55 mm, more preferably between 40 and 53 mm, most preferred between 48 and 50 mm. By choosing the coupling plate complying with these dimensions, the effect of significantly reducing the penetration depth in combination with improving the homogeneity of the ultrasonic field in the near field is achieved.

Furthermore, the piezo element is closely coupled to the coupling plate, preferably by means of a bonding layer. The bonding layer may have a thickness of about 0.2 mm and may be made from a two-component adhesive. Besides the effect of adhering the piezo element to the coupling plate, the bonding layer may also have the effect of electrically isolating the piezo element from the coupling plate.

In order to further minimize the intensity of the ultrasonic field in a depth deeper than 70 mm, preferably deeper than 40 mm, a convex acoustic lens is arranged at the coupling plate for expanding the emitted ultrasonic field, preferably a convex acoustic lens with a focal length of 80 mm at a diameter of 35 mm. By means of the convex acoustic lens the homogeneity of the field is improved and the intensity of the ultrasonic field can be further minimized in an area deeper than 70 mm below the skin.

95% or more of the energy of the ultrasonic field in an aqueous substance is accumulated in axial direction in a depth of 35% to 100% of the diameter of the piezo element starting from the coupling plate. By this relation, the depth of the ultrasonic can be limited to a region close to the surface of the coupling plate.

It has been found that the desired intensity distribution of the ultrasonic field can be achieved by means of the proper choice of material and/or dimensions of the coupling plate. Accordingly, the device of the present disclosure obviates the need for using more than one piezo element in the device, as it is customary in the prior art, in order to achieve the desired field distribution. The device according to the present disclosure also obviates the need for using acoustic focusing lenses, as it is customary in the prior art, in order to achieve the desired field distribution.

The device according to the present disclosure preferably uses a single piezo element.

The present disclosure, furthermore, relates to a method of non-invasive lipolysis by applying an ultrasonic field to human tissue, the ultrasonic field having frequencies of between 0.9 MHz and 4 MHz, the intensity of the ultrasonic field applied to the tissue being minimized in a depth deeper than 70 mm, preferably deeper than 40 mm, more preferred deeper than 30 mm.

Preferably, the ultrasonic field is applied to the tissue by means of the device specified above.

By the device of the present disclosure, it can be achieved hat the ultrasonic waves in the ultrasonic field propagate in a substantially parallel and/or divergent manner. The strong attenuation of the intensity of the field is attributed to the proper choice of the coupling plate. Accordingly, in the method the ultrasonic waves also propagate in a parallel and/or divergent manner. This may also lead to an improved homogeneity of the intensity field distribution in the areas of the higher intensity, namely in the areas near to the skin.

It has been found that the effect of the lipolysis according to the method presented herein is based on the application of a thermo-mechanical cell stressor to the adipose tissue. By means of the mechanical exposure and the thermal cell stress the permeability of the membranes of the adipocytes is changed and the incorporated proteins of the membrane are denaturated which leads to a destruction of these cells. The draining of these adipocytes takes place in a time shifted manner to the application of the mechanical and thermal cell stress.

Furthermore, it has been shown that the growth of collagenic tissue is stimulated by the application of the ultrasonic field generated by the device described above. By this effect, the device can also be used for treating cellulitis.

### BRIEF DESCRIPTION OF THE FIGURES

The invention is described using a preferred embodiment.

FIG. 1 is a schematic cross-section of the device for generating an ultrasonic field.

FIG. 2 is a schematic diagram of Fresnel and Kirchhoff's diffraction in dependency of the distance of the source.

FIG. 3 is a schematic showing the pressure distribution of sound pressure of an ultrasonic field in water according to the prior art.

FIG. 4 shows the pressure distribution according to the present disclosure.

FIG. 5 shows a schematic representation of the intensity distribution of the ultrasonic field of the present disclosure.

FIG. 6 shows a device for generating an ultrasonic field in a different embodiment including an acoustic lens.

### DETAILED DESCRIPTION OF THE FIGURES

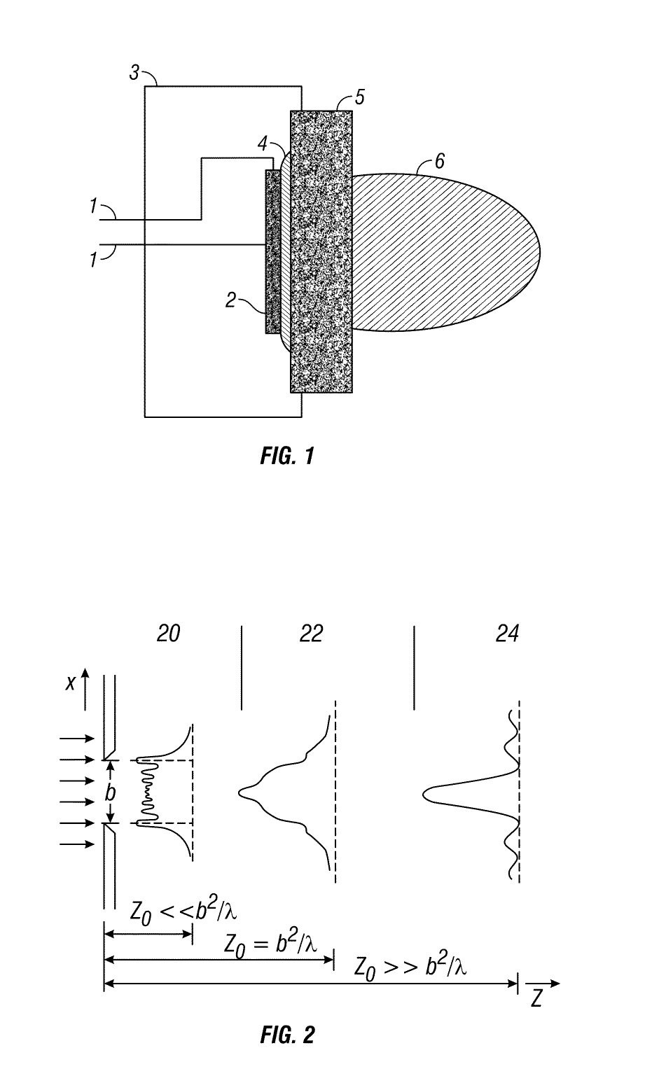

According to FIG. 1, the ultrasonic wave generator comprises a coupling plate 5 which is bonded via a bonding layer 4 to a piezo element 2. The thickness of the bonding layer 4 is about 0.2 mm but may be smaller, preferably approx. 0.025 mm. The bonding layer 4 may be made from a two-component adhesive such as “Scotch-Weld DP810” of the company 3M.

The piezo element 2 is contacted by two electrical contacts 1 which are connected to a common piezo driver. The piezo driver drives the piezo element 2 at a frequency of between 0.9 MHz and 4 MHz.

By activating the piezo element 2 via the two contacts 1 by means of the piezo driver the coupling plate 5 is caused to vibrate, emitting at the axial front of the coupling plate 5 an ultrasonic field 6. The frequency of the ultrasonic field 6 substantially corresponds to the driving frequency of the piezo element 2. The piezo element preferably shows specific dimensions, in particular the relation of the radial mode frequency to the serial mode frequency of the piezo element is set to 1.13-1.16. Furthermore, the piezo element preferably has a resonance frequency of between 0.9 and 4 MHz, corresponding to the driving frequency of the piezo driver. As it is a relatively strong piezoelectric material, the piezo element preferably is of the lead-zirconate-titanate-type (PZT).

In the medical applications contemplated in the present application, the piezo element preferably has a diameter of between 20 and 70 mm, preferably between 25 and 50 mm, more preferably between 28 and 40 mm, most preferred of 30 mm. Electronically, the piezo element preferably has a capacity of between 3,000 and 6,500 pF, preferably between 3,500 and 5,000 pF, most preferred between 3,600 and 4350 pF.

It has been found that the dimensions and/or material of the coupling plate preferably comply with the following characteristics: the coupling plate preferably comprises a synthetic material, a plastics material, a composite material and/or a metal, and is preferably made from aluminum, more preferably from aluminum 7075, the material of the coupling plate preferably having a sound velocity of between 3,000 and 6,000 m/s. Preferably the coupling plate has a thickness of between 3 and 15 mm, preferably 5 to 12 mm, more preferably between 6 and 9 mm, even more preferred between 7 and 8 mm, most preferred between 7.0 and 7.3 mm. In a preferred embodiment, the coupling plate has a circular shape and has a diameter of between 35 and 95 mm, preferably 45 and 85 mm, more preferably between 55 and 80 mm, most preferred between 60 and 75 mm. Preferably, the coupling plate has a circular shape and has an active diameter of between 25 and 60 mm, preferably between 35 and 55 mm, more preferably between 40 and 53 mm, most preferred between 48 and 50 mm.

The size and wave distribution of the sonic or ultrasonic field created by the piezo element is very complex and can hardly be calculated. It is determined by principles of diffraction. FIG. 2 shows a schematic diagram with an aperture at the left side, which is penetrated by the waves and stands for the axial surface of the coupling plate 5. Some of the waves left of the aperture pass the opening with the diameter b and cause the wave distributions shown on the right side of the aperture. The resulting field is caused according to the Kirchhoff's integral of diffraction as a superimposition of a field caused by Fresnel's diffraction with a field caused by Fraunhofer's diffraction. The Fresnel's diffraction is dominating in the near zone 20 where z₀<<b²/λ and the Fraunhofer's diffraction is dominating in a distant zone 24, where z₀>>b²/λ. b is the diameter of the coupling plate.

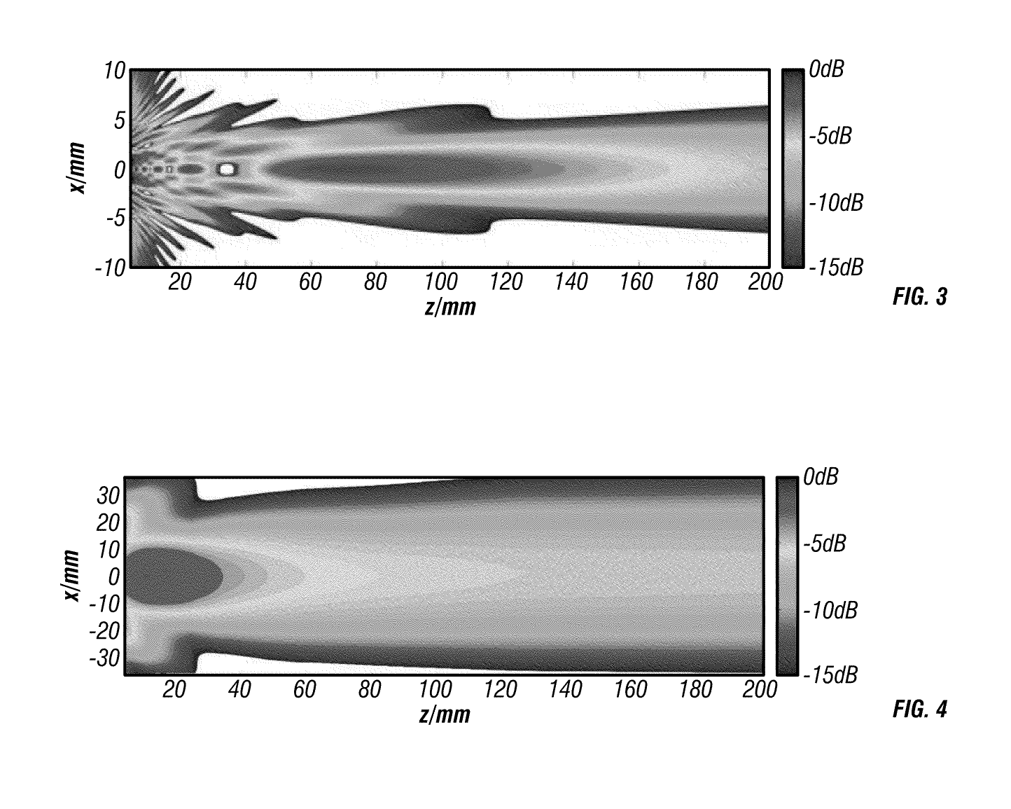

FIG. 3 shows the ultrasonic field or sound pressure distribution in water as it is known from the prior art. It shows the sound field of a non focusing 4 MHz ultrasonic transducer with a near field length of N=67 mm in water. The plot shows the sound pressure at a logarithmic db-scale. It can be seen that the intensity of the ultrasonic field has a considerable strength even at the depth of z=200 mm.

The aim of the present disclosure is to achieve an intensity distribution which shows a high and homogenous intensity in a depth up to 40 mm to 70 mm and a low intensity in a depth deeper than 40 mm to 70 mm.

It has been found that by optimizing the characteristics of the coupling plate this intensity distribution of the ultrasonic field can be achieved. In particular, different coupling plates with different diameters and thicknesses and materials were examined and tested. During the testing process the wave distribution of the ultrasonic field was scanned. The suitable parameters of the mentioned factors of influence are found, when the depth of the ultrasonic field deeper than 80 mm in z-direction is minimized.

By using the described method of optimizing the parameters a device has been designed, having optimized characteristics of the ultrasonic field distribution, as is shown in FIG. 4.

FIG. 4 shows the field measured on the basis of the following device parameters:

Piezo Type: PZT (lead-zirconate-titanate-type)

Frequency in Thickness: 1200-1225 kHz

Serial mode Frequency Fs: 73-75 kHz

Radial Mode Frequency Fp: 82.5-87 kHz

Relation Fp/Fs: 1.13-1.16

Capacity: 3600-4350 pF

Diameter of the piezo element: 28 mm

Size of the coupling plate: 48-50 mm

Outer diameter 60-75 mm

Material of the coupling plate: Aluminium 7075

Thickness of the coupling plate: 7-7.2 mm

It can be seen in FIG. 4 that the ultrasonic field is almost eliminated at a depth of z>80 mm. In this respect it is to be noted that only a single piezo element is used and no acoustic focusing lens is present in the device. Nevertheless, the strong attenuation of the ultrasonic field in the area deeper than 80 mm can be observed. In addition, the intensity distribution in the area directly below the skin, i.e. between 0 and 80 mm, is rather homogeneous, in particular when compared to the intensity distribution shown in FIG. 3.

Accordingly, it assumed that by the device of the present disclosure, it can be achieved hat the ultrasonic waves in the ultrasonic field propagate in a substantially parallel and/or divergent manner, leading to the high degree of homogeneity in the zone near to the skin. The strong attenuation of the intensity of the field is attributed to interference phenomena. The two effects are achieved by the proper choice of the dimensions and/or material of the coupling plate.

Accordingly, the device of the present disclosure obviates the need for using more than one piezo element in the device, as it is customary in the prior art, in order to achieve the desired field distribution. The device according to the present disclosure also obviates the need for using acoustic focusing lenses, as it is customary in the prior art, in order to achieve the desired field distribution.

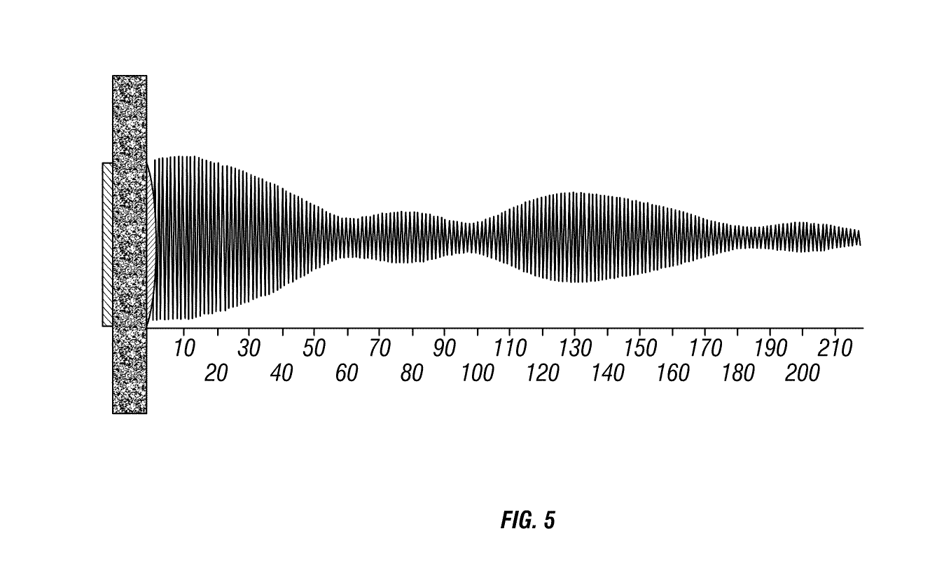

FIG. 5 offers an explanation of the effects. It has been measured that at least two different frequencies are emitted from the coupling plate 5 even though the piezo element 2 was driven at a single frequency. It is assumed that the multiple reflections of the sound at the boundary surfaces of the coupling plate and the different characteristic wave impedances are responsible for the shift. Furthermore, the waves are emitted under different phases, also due to the reflections of the acoustic wave at the boundary layers of the coupling plate, leading to destructive interferences at least in the far field. In addition, it is assumed that a Doppler shift of the reflected waves takes place because the waves are reflected on a vibrating boundary layer.

The resulting and measurable effect shown in FIG. 5 is that destructive interferences cancel the ultrasonic field in an area deeper than about 40 mm to 50 mm in the configuration described above. Furthermore, as has been shown in FIG. 4, the intensity distribution is considerably more homogenous in the area between 0 and 40 mm than in the conventional example shown in FIG. 3.

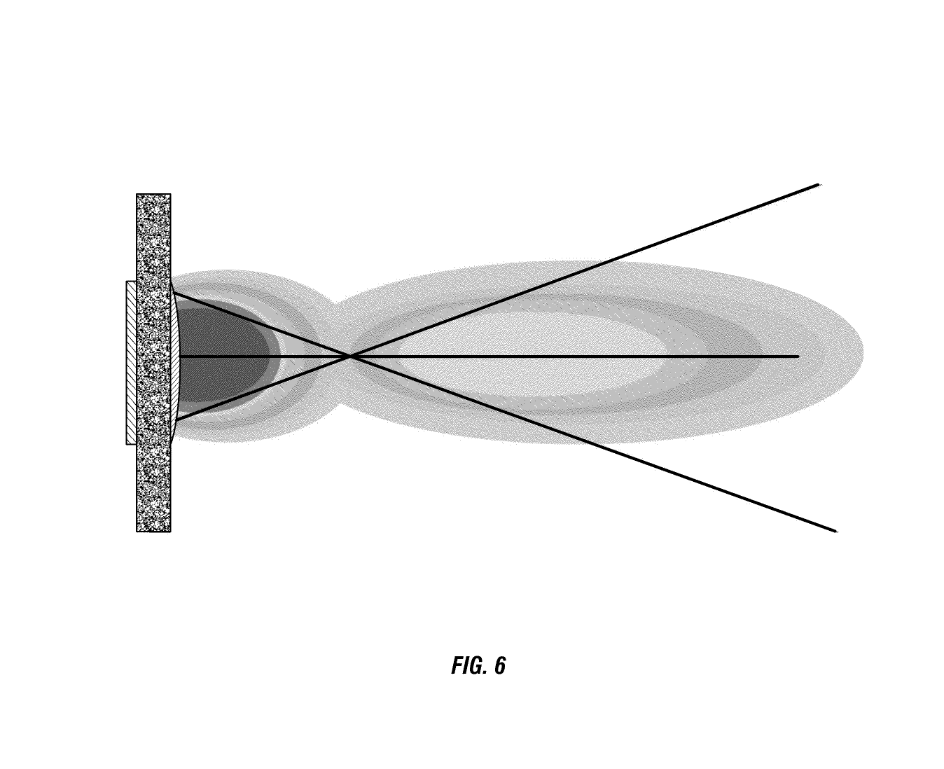

FIG. 6 shows yet another example in which an acoustic lens is placed on top of the coupling plate. The acoustic lens is intended to expand the ultrasonic field such that it becomes divergent. This divergent field shows even better characteristics as to the decay in intensity of the ultrasonic field than the field shown in FIG. 4.

Ultrasonic fields can be used for the non-invasive lipolysis. In such treatments of lipolysis the i.e. human tissue is set under vibrations in a non-invasive process through the skin and the vibrations stimulate the flow of blood in that tissue and intensify the metabolism. Thereby the fat in that tissue is reduced in a very gentle and conservative way. Also cellulitis can be reduced in this way.

1. Device for generating an ultrasonic field having frequencies between 0.9 MHz and 4 MHz in an object, preferably in water or in human tissue, the device comprising a piezo element (1) affixed to a coupling plate (5), the coupling plate emitting the ultrasonic field (6) into the object, wherein the dimensions and/or material of the coupling plate are chosen such that the intensity of the ultrasonic field (6) emitted into the object is minimized in a depth deeper than 70 mm.

2. Device according to claim 1, wherein the dimensions and/or material of the coupling plate are chosen such that the intensity of the ultrasonic field (6) emitted into the object is minimized in a depth deeper than 40 mm, preferably in a depth deeper than 30 mm.

3. Device according to claim 1, wherein the diameter of the coupling plate, the thickness of the coupling plate and/or the material of the coupling plate are dimensioned such that the intensity of the ultrasonic field emitted into the object is minimized in a depth deeper than 70 mm, preferably in a depth deeper than 40 mm, more preferred in a depth deeper than 30 mm.

4. Device according to claim 1, wherein the relation of the radial mode frequency to the serial mode frequency of the piezo element is set to 1.13-1.16, and/or wherein the piezo element has a resonance frequency of between 0.9 and 4 MHz, and/or wherein the piezo element has a diameter of between 20 and 70 mm, preferably between 25 and 50 mm, more preferably between 28 and 40 mm, most preferred of 30 mm, and/or wherein the piezo element has a capacity of between 3,000 and 6,500 pF, preferably between 3,500 and 5,000 pF, most preferred between 3,600 and 4350 pF and/or wherein the piezo element is of the lead-zirconate-titanate-type (PZT).

5. Device according to claim 1, wherein the coupling plate is dimensioned such that at least two different frequencies are emitted from the coupling plate into the object when the piezo element is driven at a single frequency, the at least two frequencies emitted by the coupling plate preferably having a frequency difference of 5 to 100 kHz, more preferably a frequency difference of 10 to 50 kHz.

6. Device according to claim 1, wherein the coupling plate comprises a synthetic material, a plastics material, a composite material and/or a metal, and is preferably made from aluminum, more preferably from aluminum 7075, the material of the coupling plate preferably having a sound velocity of between 3,000 and 6,000 m/s.

7. Device according to claim 1, wherein the coupling plate has a thickness of between 3 and 15 mm, preferably 5 to 12 mm, more preferably between 6 and 9 mm, even more preferred between 7 and 8 mm, most preferred between 7.0 and 7.3 mm, and/or wherein the coupling plate has a circular shape and has a diameter of between 35 and 95 mm, preferably 45 and 85 mm, more preferably between 55 and 80 mm, most preferred between 60 and 75 mm, and/or wherein the coupling plate has a circular shape and has an active diameter of between 25 and 60 mm, preferably between 35 and 55 mm, more preferably between 40 and 53 mm, most preferred between 48 and 50 mm.

8. Device according to claim 1, wherein the piezo element is closely coupled to the coupling plate, preferably by means of a bonding layer (4) which preferably has a thickness of about 0.2 mm and which preferably is made from a two-component adhesive.

9. Device according to claim 1, wherein a convex acoustic lens is arranged at the coupling plate for expanding the emitted ultrasonic field, preferably a convex acoustic lens with a focal length of 80 mm at a diameter of 35 mm.

10. Device according to claim 1, wherein at least 95% of the energy of the ultrasonic field in an aqueous substance is accumulated in axial direction in a depth of 35% to 100% of the diameter of the piezo element, starting from the coupling plate (5).

11. Device according to claim 1, wherein the dimensions and/or material of the coupling plate are chosen such that the intensity of the ultrasonic field is homogenized in the object in an area between the coupling plate and the depth in which the ultrasonic field is minimized, preferably in a depth less than 70 mm, more preferably in a depth less than 40 mm, even more preferred in a depth less than 30 mm.

12. Device according to claim 1, wherein the device uses a single piezo element.

13. Method of non-invasive lipolysis by applying an ultrasonic field to human tissue, the ultrasonic field having frequencies of between 0.9 MHz and 4 MHz, the intensity of the ultrasonic field applied to the tissue being minimized in a depth deeper than 70 mm, preferably deeper than 40 mm, more preferred deeper than 30 mm.

14. Method according to claim 13, wherein the ultrasonic waves in the ultrasonic field propagate in a substantially parallel and/or divergent manner.

15. Method according to claim 13, wherein the ultrasonic field is applied to the tissue by means of a device according to any one of claims 1 to 12, preferably using a single piezo element.
